(19) United States
(12) Patent Application Publication
(10) Pub. No.: US 2010/0168851 A1
(43) Pub. Date: Jul. 1, 2010

(54) Surface Modified Biomedical Devices

(76) Inventors: David Paul Vanderbilt, Webster, NY (US); Paul L. Valint, JR., Pittsford, NY (US); Joseph A. McGee, Canandaigua, NY (US)

Correspondence Address: Bausch & Lomb Incorporated, One Bausch & Lomb Place, Rochester, NY, 14604-2701, US

(21) Appl. No.: 12/641,412

(22) Filed: Dec. 18, 2009

Related U.S. Application Data
(60) Provisional application No. 61/203,881, filed on Dec. 30, 2008.

(51) Int. Cl. A61F 2/16; B05D 7/00; B65B 55/02; G02C 7/04

(52) U.S. Cl. 623/6.62; 427/2.1; 53/425; 351/160 H

(57) ABSTRACT

Disclosed are surface modified biomedical devices having a coating on a surface thereof, the coating comprising an inner layer comprising a polymer comprising monomeric units derived from an ethylenically unsaturated monomer containing a boronic acid moiety, and an outer layer comprising a hydrophilic hydrolyzed reactive polymer comprising monomeric units derived from an ethylenically unsaturated containing monomer having hydrolyzable reactive functionalities.

This application claims the benefit of Provisional Patent Application No. 61/203,881 filed Dec. 30, 2008 which is incorporated by reference herein.

### BACKGROUND OF THE INVENTION

1. Technical Field

The present invention generally relates to surface modified biomedical devices such as contact lenses, intraocular lenses, and other ophthalmic devices.

2. Description of the Related Art

Medical devices such as ophthalmic lenses made from, for example, silicone-containing materials, have been investigated for a number of years. Such materials can generally be subdivided into two major classes, namely, hydrogels and non-hydrogels. Hydrogels can absorb and retain water in an equilibrium state, whereas non-hydrogels do not absorb appreciable amounts of water. Regardless of their water content, both hydrogel and non-hydrogel silicone medical devices tend to have relatively hydrophobic, non-wettable surfaces that have a high affinity for lipids. This problem is of particular concern with contact lenses.

Those skilled in the art have long recognized the need for modifying the surface of such silicone contact lenses so that they are compatible with the eye. It is known that increased hydrophilicity of the lens surface improves the wettability of the contact lens. This, in turn, is associated with improved wear comfort of contact lenses. Additionally, the surface of the lens can affect the lens's susceptibility to deposition, particularly the deposition of proteins and lipids resulting from tear fluid during lens wear. Accumulated deposition can cause eye discomfort or even inflammation. In the case of extended wear lenses (i.e., lenses used without daily removal of the lens before sleep), the surface is especially important, since extended wear lenses must be designed for high standards of comfort and biocompatibility over an extended period of time.

Silicone lenses have been subjected to plasma surface treatment to improve their surface properties, e.g., surfaces have been rendered more hydrophilic, deposit resistant, scratch-resistant, or otherwise modified. Examples of previously disclosed plasma surface treatments include subjecting the surface of a contact lens to a plasma containing an inert gas or oxygen (see, for example, U.S. Pat. Nos. 4,055,378; 4,122,942; and 4,214,014); various hydrocarbon monomers (see, for example, U.S. Pat. No. 4,143,949); and combinations of oxidizing agents and hydrocarbons such as water and ethanol (see, for example, WO 95/04609 and U.S. Pat. No. 4,632,844). U.S. Pat. No. 4,312,575 discloses a process for providing a barrier coating on a silicone or polyurethane lens by subjecting the lens to an electrical glow discharge (plasma) process conducted by first subjecting the lens to a hydrocarbon atmosphere followed by subjecting the lens to oxygen during flow discharge, thereby increasing the hydrophilicity of the lens surface.

U.S. Pat. No. 6,582,754 (“the '754 patent”) discloses a process for coating a material surface involving the steps of (a) providing an organic bulk material having functional groups on its surface; (b) covalently binding to the surface of the bulk material a layer of a first compound having a first reactive group and an ethylenically unsaturated double bond by reacting the function groups on the surface of the bulk material with the first reactive group of the first compound; (c) copolymerizing, on the surface of the bulk material, a first hydrophilic monomer and a monomer comprising a second reactive group to form a coating comprising a plurality of primary polymer chains which are covalently bonded to the surface through the first compound, wherein each primary polymer chain comprises second reactive; (d) reacting the second reactive groups of the primary polymer chains with a second compound comprising an ethylenically unsaturated double bond and a third reactive group that is co-reactive with the second reactive group, to covalently bind the second compound to the primary polymer chains; and (e) graft-polymerizing a second hydrophilic monomer to obtain a branched hydrophilic coating on the surface of the bulk material, wherein the branched hydrophilic coating comprises the plurality of the primary polymer chains and a plurality of secondary chains each of which is covalently attached through the second compound to one of the primary chains. The process disclosed in the '754 patent is time consuming as it involves multiple steps and uses many reagents in producing the coating on the substrate.

U.S. Patent Application Publication No. 20080151181 (“the '181 application), commonly assigned to assignee herein Bausch & Lomb Incorporated, discloses a contact lens having its surfaces coated with an inner layer and an outer layer, the inner layer comprising a polymer comprising monomeric units derived from an ethylenically unsaturated monomer containing a boronic acid moiety, and the outer layer comprising a diol. The '181 application further discloses that the diol layer includes at least one diol-terminated polymer member selected from the group consisting of diol-terminated polyvinyl pyrrolidinone, diol-terminated polyacrylamides, diol-terminated polyethylene oxides, and diol-terminated polyethylene oxide (PEO)/polypropylene oxide (PPO) block copolymers.

It would be desirable to provide improved methods for surface treating a biomedical device such as a contact lens to obtain a surface modified biomedical device with an optically clear, hydrophilic surface film that will not only exhibit improved wettability and lubriciousness, but which may generally allow the use of the device in the human eye for an extended period of time.

### SUMMARY OF THE INVENTION

In accordance with one embodiment of the present invention, a surface modified biomedical device having a coating on a surface thereof is provided, the coating comprising an inner layer comprising a polymer comprising monomeric units derived from an ethylenically unsaturated monomer containing one or more boronic acid moieties, and an outer layer comprising a hydrophilic hydrolyzed reactive polymer comprising monomeric units derived from an ethylenically unsaturated-containing monomer having hydrolyzable reactive functionalities.

In accordance with a second embodiment of the present invention, a method for making a surface modified biomedical device is provided, the method comprising exposing a biomedical device having a plurality of biomedical device surface functional groups to (a) one or more polymers comprising monomeric units derived from an ethylenically unsaturated monomer containing one or more boronic acid moieties and; and (b) a hydrophilic hydrolyzed reactive polymer comprising monomeric units derived from an ethylenically unsaturated-containing monomer having hydrolyzable reactive functionalities, thus forming a biocompatible coating on the surface on the biomedical device.

The surface modified biomedical devices of the present invention are believed to provide a higher level of performance quality and/or comfort to the users due to their hydrophilic or lubricious (or both) surfaces. Hydrophilic and/or lubricious surfaces of the biomedical devices herein such as contact lenses substantially prevent or limit the adsorption of tear lipids and proteins on, and their eventual absorption into, the lenses, thus preserving the clarity of the contact lenses. This, in turn, preserves their performance quality thereby providing a higher level of comfort to the wearer.

### DESCRIPTION OF THE PREFERRED EMBODIMENTS

The present invention is directed to surface modified biomedical devices. As used herein, the term “biomedical device” shall be understood to mean any article that is designed to be used while either in or on mammalian tissues or fluid, and preferably in or on human tissue or fluids. Representative examples of biomedical devices include, but are not limited to, artificial ureters, diaphragms, intrauterine devices, heart valves, catheters, denture liners, prosthetic devices, ophthalmic lens applications, where the lens is intended for direct placement in or on the eye, such as, for example, intraocular devices and contact lenses. The preferred biomedical devices are ophthalmic devices, particularly contact lenses, and most particularly contact lenses made from silicone hydrogels.

As used herein, the term “ophthalmic device” refers to devices that reside in or on the eye. These devices can provide optical correction, wound care, drug delivery, diagnostic functionality or cosmetic enhancement or effect or a combination of these properties. Useful ophthalmic devices include, but are not limited to, ophthalmic lenses such as soft contact lenses, e.g., a soft, hydrogel lens; soft, non-hydrogel lens and the like, hard contact lenses, e.g., a hard, gas permeable lens material and the like, intraocular lenses, overlay lenses, ocular inserts, optical inserts and the like. As is understood by one skilled in the art, a lens is considered to be “soft” if it can be folded back upon itself without breaking.

The biomedical devices to be surface modified according to the present invention can be any material known in the art capable of forming a biomedical device as described above. In one embodiment, a biomedical device includes devices formed from material not hydrophilic per se. Such devices are formed from materials known in the art and include, by way of example, polysiloxanes, perfluoropolyethers, fluorinated poly(meth)acrylates or equivalent fluorinated polymers derived, e.g., from other polymerizable carboxylic acids, polyalkyl(meth)acrylates or equivalent alkylester polymers derived from other polymerizable carboxylic acids, or fluorinated polyolefins, such as fluorinated ethylene propylene polymers, or tetrafluoroethylene, preferably in combination with a dioxol, e.g., perfluoro-2,2-dimethyl-1,3-dioxol. Representative examples of suitable bulk materials include, but are not limited to, Lotrafilcon A, Neofocon, Pasifocon, Telefocon, Silafocon, Fluorsilfocon, Paflufocon, Silafocon, Elastofilcon, Fluorofocon or Teflon AF materials, such as Teflon AF 1600 or Teflon AF 2400 which are copolymers of about 63 to about 73 mol % of perfluoro-2,2-dimethyl-1,3-dioxol and about 37 to about 27 mol % of tetrafluoroethylene, or of about 80 to about 90 mol % of perfluoro-2,2-dimethyl-1,3-dioxol and about 20 to about 10 mol % of tetrafluoroethylene.

In another embodiment, a biomedical device includes devices formed from material hydrophilic per se, since reactive groups, e.g., carboxy, carbamoyl, sulfate, sulfonate, phosphate, amine, ammonium or hydroxy groups, are inherently present in the material and therefore also at the surface of a biomedical device manufactured therefrom. Such devices are formed from materials known in the art and include, by way of example, polyhydroxyethyl acrylate, polyhydroxyethyl methacrylate, polyvinyl pyrrolidone (PVP), polyacrylic acid, polymethacrylic acid, polyacrylamide, polydimethylacrylamide (DMA), polyvinyl alcohol and the like and copolymers thereof, e.g., from two or more monomers selected from hydroxyethyl acrylate, hydroxyethyl methacrylate, N-vinyl pyrrolidone, acrylic acid, methacrylic acid, acrylamide, dimethyl acrylamide, vinyl alcohol and the like. Representative examples of suitable bulk materials include, but are not limited to, Polymacon, Tefilcon, Methafilcon, Deltafilcon, Bufilcon, Phemfilcon, Ocufilcon, Focofilcon, Etafilcon, Hefilcon, Vifilcon, Tetrafilcon, Perfilcon, Droxifilcon, Dimefilcon, Isofilcon, Mafilcon, Nelfilcon, Atlafilcon and the like. Examples of other suitable bulk materials include Balafilcon A, Hilafilcon A, Alphafilcon A, Bilafilcon B and the like.

In another embodiment, biomedical devices to be surface modified according to the present invention include devices which are formed from material which are amphiphilic segmented copolymers containing at least one hydrophobic segment and at least one hydrophilic segment which are linked through a bond or a bridge member.

It is particularly useful to employ biocompatible materials herein including both soft and rigid materials commonly used for ophthalmic lenses, including contact lenses. In general, non-hydrogel materials are hydrophobic polymeric materials that do not contain water in their equilibrium state. Typical non-hydrogel materials comprise silicone acrylics, such as those formed bulky silicone monomer (e.g., tris(trimethylsiloxy)silylpropyl methacrylate, commonly known as “TRIS” monomer), methacrylate end-capped poly(dimethylsiloxane) prepolymer, or silicones having fluoroalkyl side groups (polysiloxanes are also commonly known as silicone polymers).

On the other hand, hydrogel materials comprise hydrated, cross-linked polymeric systems containing water in an equilibrium state. Hydrogel materials contain about 5 weight percent water or more (up to, for example, about 80 weight percent). The preferred hydrogel materials, include silicone hydrogel materials. In one preferred embodiment, materials include vinyl functionalized polydimethylsiloxanes copolymerized with hydrophilic monomers as well as fluorinated methacrylates and methacrylate functionalized fluorinated polyethylene oxides copolymerized with hydrophilic monomers. Representative examples of suitable materials for use herein include those disclosed in U.S. Pat. Nos. 5,310,779; 5,387,662; 5,449,729; 5,512,205; 5,610,252; 5,616,757; 5,708,094; 5,710,302; 5,714,557 and 5,908,906, the contents of which are incorporated by reference herein.

In one embodiment, hydrogel materials for biomedical devices, such as contact lenses, can contain a hydrophilic monomer such as one or more unsaturated carboxylic acids, vinyl lactams, amides, polymerizable amines, vinyl carbonates, vinyl carbamates, oxazolone monomers, copolymers thereof and the like and mixtures thereof. Useful amides include acrylamides such as N,N-dimethylacrylamide and N,N-dimethylmethacrylamide. Useful vinyl lactams include cyclic lactams such as N-vinyl-2-pyrrolidone. Examples of other hydrophilic monomers include hydrophilic prepolymers such as poly(alkene glycols) functionalized with polymerizable groups. Examples of useful functionalized poly(alkene glycols) include poly(diethylene glycols) of varying chain length containing monomethacrylate or dimethacrylate end caps. In a preferred embodiment, the poly(alkene glycol) polymer contains at least two alkene glycol monomeric units. Still further examples are the hydrophilic vinyl carbonate or vinyl carbamate monomers disclosed in U.S. Pat. No. 5,070,215, and the hydrophilic oxazolone monomers disclosed in U.S. Pat. No. 4,910,277. Other suitable hydrophilic monomers will be apparent to one skilled in the art. In another embodiment, a hydrogel material can contain a siloxane-containing monomer and at least one of the aforementioned hydrophilic monomers and/or prepolymers.

Non-limited examples of hydrophobic monomers are C₁-C₂₀ alkyl and C₃-C₂₀ cycloalkyl(meth)acrylates, substituted and unsubstituted aryl(meth)acrylates (wherein the aryl group comprises 6 to 36 carbon atoms), (meth) acrylonitrile, styrene, lower alkyl styrene, lower alkyl vinyl ethers, and C₂-C₁₀ perfluoroalkyl(meth)acrylates and correspondingly partially fluorinate (meth)acrylates.

A wide variety of materials can be used herein, and silicone hydrogel contact lens materials are particularly preferred. Silicone hydrogels generally have a water content greater than about 5 weight percent and more commonly between about 10 to about 80 weight percent. Such materials are usually prepared by polymerizing a mixture containing at least one silicone-containing monomer and at least one hydrophilic monomer. Typically, either the silicone-containing monomer or the hydrophilic monomer functions as a crosslinking agent (a crosslinker being defined as a monomer having multiple polymerizable functionalities) or a separate crosslinker may be employed. Applicable silicone-containing monomers for use in the formation of silicone hydrogels are well known in the art and numerous examples are provided in U.S. Pat. Nos. 4,136,250; 4,153,641; 4,740,533; 5,034,461; 5,070,215; 5,260,000; 5,310,779; and 5,358,995.

Representative examples of applicable silicon-containing monomers include bulky polysiloxanylalkyl(meth)acrylic monomers. An example of a bulky polysiloxanylalkyl(meth)acrylic monomer is represented by the structure of Formula I:

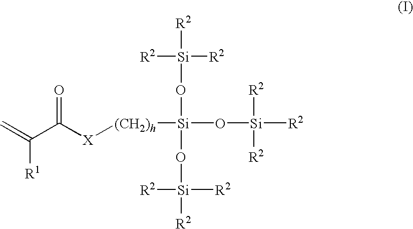

wherein X denotes —O— or —NR— wherein R denotes hydrogen or a C₁-C₄ alkyl; each R¹ independently denotes hydrogen or methyl; each R² independently denotes a lower alkyl radical, phenyl radical or a group represented by

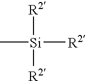

wherein each R^{2′} independently denotes a lower alkyl or phenyl radical; and h is 1 to 10.

Representative examples of other applicable silicon-containing monomers includes, but are not limited to, bulky polysiloxanylalkyl carbamate monomers as generally depicted in Formula Ia:

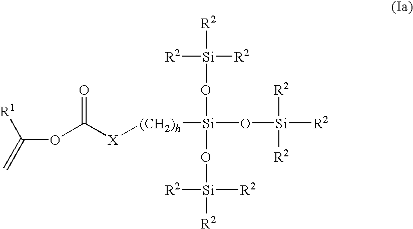

wherein X denotes —NR—; wherein R denotes hydrogen or a C₁-C₄ alkyl; R¹ denotes hydrogen or methyl; each R² independently denotes a lower alkyl radical, phenyl radical or a group represented by

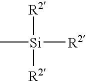

wherein each R^{2′} independently denotes a lower alkyl or phenyl radical; and h is 1 to 10, and the like.

Examples of bulky monomers are 3-methacryloyloxypropyltris(trimethyl-siloxy)silane or tris(trimethylsiloxy)silylpropyl methacrylate, sometimes referred to as TRIS and tris(trimethylsiloxy)silylpropyl vinyl carbamate, sometimes referred to as TRIS-VC and the like and mixtures thereof.

Such bulky monomers may be copolymerized with a silicone macromonomer, which is a poly(organosiloxane) capped with an unsaturated group at two or more ends of the molecule. U.S. Pat. No. 4,153,641 discloses, for example, various unsaturated groups such as acryloxy or methacryloxy groups.

Another class of representative silicone-containing monomers includes, but is not limited to, silicone-containing vinyl carbonate or vinyl carbamate monomers such as, for example, 1,3-bis[4-vinyloxycarbonyloxy)but-1-yl]tetramethyl-disiloxane; 3-(trimethylsilyl)propyl vinyl carbonate; 3-(vinyloxycarbonylthio)propyl-[tris(trimethylsiloxy)silane]; 3-[tris(trimethylsiloxy)silyl]propyl vinyl carbamate; 3-[tris(trimethylsiloxy)silyl]propyl allyl carbamate; 3-[tris(trimethylsiloxy)silyl]propyl vinyl carbonate; t-butyldimethylsiloxyethyl vinyl carbonate; trimethylsilylethyl vinyl carbonate; trimethylsilylmethyl vinyl carbonate and the like and mixtures thereof.

Another class of silicon-containing monomers includes polyurethane-polysiloxane macromonomers (also sometimes referred to as prepolymers), which may have hard-soft-hard blocks like traditional urethane elastomers. They may be end-capped with a hydrophilic monomer such as HEMA. Examples of such silicone urethanes are disclosed in a variety or publications, including Lai, Yu-Chin, “The Role of Bulky Polysiloxanylalkyl Methacryates in Polyurethane-Polysiloxane Hydrogels,” Journal of Applied Polymer Science, Vol. 60, 1193-1199 (1996). PCT Published Application No. WO 96/31792 discloses examples of such monomers, which disclosure is hereby incorporated by reference in its entirety. Further examples of silicone urethane monomers are represented by Formulae II and III:

E(*D*A*D*G)ₐ*D*A*D*E′; or (II)

E(*D*G*D*A)ₐ*D*A*D*E′; or (III)

wherein:

D independently denotes an alkyl diradical, an alkyl cycloalkyl diradical, a cycloalkyl diradical, an aryl diradical or an alkylaryl diradical having 6 to about 30 carbon atoms;

G independently denotes an alkyl diradical, a cycloalkyl diradical, an alkyl cycloalkyl diradical, an aryl diradical or an alkylaryl diradical having 1 to about 40 carbon atoms and which may contain ether, thio or amine linkages in the main chain;

* denotes a urethane or ureido linkage;

a is at least 1;

A independently denotes a divalent polymeric radical of Formula IV:

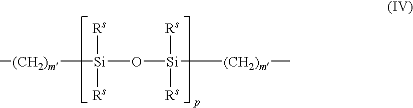

wherein each R^{s} independently denotes an alkyl or fluoro-substituted alkyl group having 1 to about 10 carbon atoms which may contain ether linkages between the carbon atoms; m′ is at least 1; and p is a number that provides a moiety weight of about 400 to about 10,000;

each of E and E′ independently denotes a polymerizable unsaturated organic radical represented by Formula V:

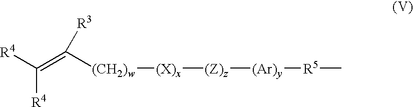

wherein: R³ is hydrogen or methyl;
R⁴ is hydrogen, an alkyl radical having 1 to 6 carbon atoms, or a —CO—Y—R⁶ radical wherein Y is —O—, —S— or —NH—;
R⁵ is a divalent alkylene radical having 1 to about 10 carbon atoms;
R⁶ is a alkyl radical having 1 to about 12 carbon atoms;
X denotes —CO— or —OCO—;
Z denotes —O— or —NH—;
Ar denotes an aromatic radical having about 6 to about 30 carbon atoms;
w is 0 to 6; x is 0 or 1; y is 0 or 1; and z is 0 or 1.

A preferred silicone-containing urethane monomer is represented by Formula VI:

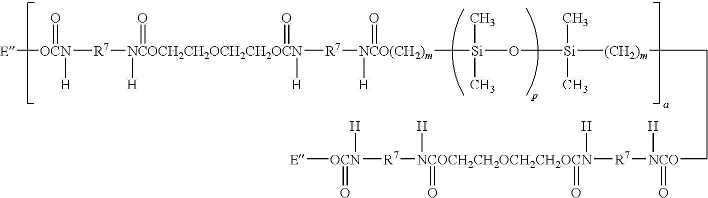

wherein m is at least 1 and is preferably 3 or 4, a is at least 1 and preferably is 1, p is a number which provides a moiety weight of about 400 to about 10,000 and is preferably at least about 30, R⁷ is a diradical of a diisocyanate after removal of the isocyanate group, such as the diradical of isophorone diisocyanate, and each E″ is a group represented by:

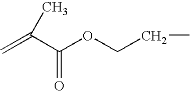

In another embodiment of the present invention, a silicone hydrogel material comprises (in bulk, that is, in the monomer mixture that is copolymerized) about 5 to about 50 percent, and preferably about 10 to about 25, by weight of one or more silicone macromonomers, about 5 to about 75 percent, and preferably about 30 to about 60 percent, by weight of one or more polysiloxanylalkyl(meth)acrylic monomers, and about 10 to about 50 percent, and preferably about 20 to about 40 percent, by weight of a hydrophilic monomer. In general, the silicone macromonomer is a poly(organosiloxane) capped with an unsaturated group at two or more ends of the molecule. In addition to the end groups in the above structural formulas, U.S. Pat. No. 4,153,641 discloses additional unsaturated groups, including acryloxy or methacryloxy. Fumarate-containing materials such as those disclosed in U.S. Pat. Nos. 5,310,779; 5,449,729 and 5,512,205 are also useful substrates in accordance with the invention. The silane macromonomer may be a silicon-containing vinyl carbonate or vinyl carbamate or a polyurethane-polysiloxane having one or more hard-soft-hard blocks and end-capped with a hydrophilic monomer.

Another class of representative silicone-containing monomers includes fluorinated monomers. Such monomers have been used in the formation of fluorosilicone hydrogels to reduce the accumulation of deposits on contact lenses made therefrom, as disclosed in, for example, U.S. Pat. Nos. 4,954,587; 5,010,141 and 5,079,319. Also, the use of silicone-containing monomers having certain fluorinated side groups, i.e., —(CF₂)—H, have been found to improve compatibility between the hydrophilic and silicone-containing monomeric units. See, e.g., U.S. Pat. Nos. 5,321,108 and 5,387,662.

The above silicone materials are merely exemplary, and other materials for use as substrates that can benefit by being coated with the hydrophilic coating composition according to the present invention and have been disclosed in various publications and are being continuously developed for use in contact lenses and other medical devices can also be used. For example, a biomedical device can be formed from at least a cationic monomer such as cationic silicone-containing monomer or cationic fluorinated silicone-containing monomers.

Contact lenses for application of the present invention can be manufactured employing various conventional techniques, to yield a shaped article having the desired posterior and anterior lens surfaces. Spincasting methods are disclosed in U.S. Pat. Nos. 3,408,429 and 3,660,545; and static casting methods are disclosed in U.S. Pat. Nos. 4,113,224, 4,197,266 and 5,271,876. Curing of the monomeric mixture may be followed by a machining operation in order to provide a contact lens having a desired final configuration. As an example, U.S. Pat. No. 4,555,732 discloses a process in which an excess of a monomeric mixture is cured by spincasting in a mold to form a shaped article having an anterior lens surface and a relatively large thickness. The posterior surface of the cured spincast article is subsequently lathe cut to provide a contact lens having the desired thickness and posterior lens surface. Further machining operations may follow the lathe cutting of the lens surface, for example, edge-finishing operations.

Typically, an organic diluent is included in the initial monomeric mixture in order to minimize phase separation of polymerized products produced by polymerization of the monomeric mixture and to lower the glass transition temperature of the reacting polymeric mixture, which allows for a more efficient curing process and ultimately results in a more uniformly polymerized product. Sufficient uniformity of the initial monomeric mixture and the polymerized product is of particular importance for silicone hydrogels, primarily due to the inclusion of silicone-containing monomers which may tend to separate from the hydrophilic comonomer.

Suitable organic diluents include, for example, monohydric alcohols such as C₆-C₁₀ straight-chained aliphatic monohydric alcohols, e.g., n-hexanol and n-nonanol; diols such as ethylene glycol; polyols such as glycerin; ethers such as diethylene glycol monoethyl ether; ketones such as methyl ethyl ketone; esters such as methyl enanthate; and hydrocarbons such as toluene. Preferably, the organic diluent is sufficiently volatile to facilitate its removal from a cured article by evaporation at or near ambient pressure.

Generally, the diluent may be included at about 5 to about 60 percent by weight of the monomeric mixture, with about 10 to about 50 percent by weight being especially preferred. If necessary, the cured lens may be subjected to solvent removal, which can be accomplished by evaporation at or near ambient pressure or under vacuum. An elevated temperature can be employed to shorten the time necessary to evaporate the diluent.

Following removal of the organic diluent, the lens can then be subjected to mold release and optional machining operations. The machining step includes, for example, buffing or polishing a lens edge and/or surface. Generally, such machining processes may be performed before or after the article is released from a mold part. As an example, the lens may be dry released from the mold by employing vacuum tweezers to lift the lens from the mold.

As one skilled in the art will readily appreciate, biomedical device surface functional groups of the biomedical device according to the present invention may be inherently present at the surface of the device. However, if the biomedical device contains too few or no functional groups, the surface of the device can be modified by known techniques, for example, plasma chemical methods (see, for example, WO 94/06485), or conventional functionalization with groups such as —OH, —NH₂ or —CO₂H. Suitable biomedical device surface functional groups of the biomedical device include a wide variety of groups well known to the skilled artisan. Representative examples of such functional groups include, but are not limited to, hydroxy groups, cis 1,2-diols, cis 1,3-diols, α hydroxy acid groups (e.g., sialic acid, salicylic acid), carboxylic acids, di-carboxylic acids, catechols, silanols, silicates and the like. For example, a biomedical device such as a silicone hydrogel formulation containing hydrophilic polymers is subjected to an oxidative surface treatment as known in the art to form at least silicates on the surface of the lens. The lens is then surface treated to form a coating on the surface thereof.

The foregoing biomedical devices can be surface modified by exposing a biomedical device having a plurality of biomedical device surface functional groups to (a) one or more polymers comprising monomeric units derived from an ethylenically unsaturated monomer containing one or more boronic acid moieties; and (b) a hydrophilic hydrolyzed reactive polymer comprising monomeric units derived from an ethylenically unsaturated-containing monomer having hydrolyzable reactive functionalities, thus forming an inner layer comprising the boronic acid-containing polymer and an outer layer comprising the hydrophilic hydrolyzed reactive polymer on the surface of the biomedical device.

Representative examples of suitable ethylenically unsaturated monomers containing one or more boronic acid moieties include ethylenically unsaturated-containing alkyl boronic acids; ethylenically unsaturated-containing cycloalkyl boronic acids; ethylenically unsaturated-containing aryl boronic acids and the like and mixtures thereof. Preferred ethylenically unsaturated monomers having one or more boronic acid moieties include 4-vinylphenylboronic acid, 3-methacrylamidophenylboronic acid and mixtures thereof.

Representative examples of alkyl groups for use herein include, by way of example, a straight or branched hydrocarbon chain radical containing carbon and hydrogen atoms of from 1 to about 18 carbon atoms with or without unsaturation, to the rest of the molecule, e.g., methyl, ethyl, n-propyl, 1-methylethyl(isopropyl), n-butyl, n-pentyl, etc., and the like.

Representative examples of cycloalkyl groups for use herein include, by way of example, a substituted or unsubstituted non-aromatic mono or multicyclic ring system of about 3 to about 24 carbon atoms such as, for example, cyclopropyl, cyclobutyl, cyclopentyl, cyclohexyl, perhydronapththyl, adamantyl and norbornyl groups bridged cyclic group or sprirobicyclic groups, e.g., sprio-(4,4)-non-2-yl and the like, optionally containing one or more heteroatoms, e.g., O and N, and the like.

Representative examples of aryl groups for use herein include, by way of example, a substituted or unsubstituted monoaromatic or polyaromatic radical containing from about 5 to about 30 carbon atoms such as, for example, phenyl, naphthyl, tetrahydronapthyl, indenyl, biphenyl and the like, optionally containing one or more heteroatoms, e.g., O and N, and the like.

Representative examples of the ethylenically unsaturated moiety of the ethylenically unsaturated monomer include, by way of example, (meth)acrylate-containing radicals, (meth)acrylamido-containing radicals, vinylcarbonate-containing radicals, vinylcarbamate-containing radicals, styrene-containing radicals, itaconate-containing radicals, vinyl-containing radicals, vinyloxy-containing radicals, fumarate-containing radicals, maleimide-containing radicals, vinylsulfonyl radicals and the like. As used herein, the term “(meth)” denotes an optional methyl substituent. Thus, for example, terms such as “(meth)acrylate” denotes either methacrylate or acrylate, and “(meth)acrylamide” denotes either methacrylamide or acrylamide.

In one embodiment, an ethylenically unsaturated moiety of the ethylenically unsaturated monomer is represented by the general formula:

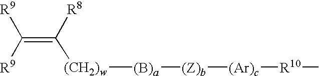

wherein R⁸ is hydrogen or a alkyl group having 1 to 6 carbon atoms such as methyl; each R⁹ is independently hydrogen, an alkyl radical having 1 to 6 carbon atoms, or a —CO—Y—R¹¹ radical wherein Y is —O—, —S— or —NH— and R¹¹ is an alkyl radical having 1 to about 10 carbon atoms; R¹⁰ is a linking group (e.g., a divalent alkenyl radical having 1 to about 12 carbon atoms); B denotes —O— or —NH—; Z denotes —CO—, —OCO— or —COO—; Ar denotes an aromatic radical having 6 to about 30 carbon atoms; w is 0 to 6; a is 0 or 1; b is 0 or 1; and c is 0 or 1. The polymerizable ethylenically unsaturated-containing radicals can be attached to the boronic acid-containing monomers as pendent groups, terminal groups or both.

In one embodiment, the polymerizable monomer containing a boronic acid moiety may further contain an electron withdrawing moiety. As used herein, the term “electron withdrawing moiety” refers to a group which has a greater electron withdrawing effect than hydrogen. A variety of electron-withdrawing moieties are known and include, by way of example, halogens (e.g., fluoro, chloro, bromo, and iodo groups), NO₂, NR₃⁺, CN, COOH(R), CF₃, and the like. The pH of the boronic acid-containing monomer can be adjusted by placing the electron withdrawing moiety in, e.g., a position meta to the boronic acid moiety on the phenyl ring. A representative example of such a boronic acid-containing monomer is represented by the general formula:

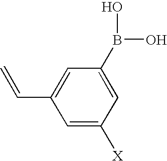

wherein X is an electron withdrawing group such as —CF₃, —NO₂, —F, —Cl or —Br.

The polymerizable monomers containing a boronic acid moiety and an electron withdrawing moiety can be prepared by the general reaction sequences set forth in Schemes I and II below:

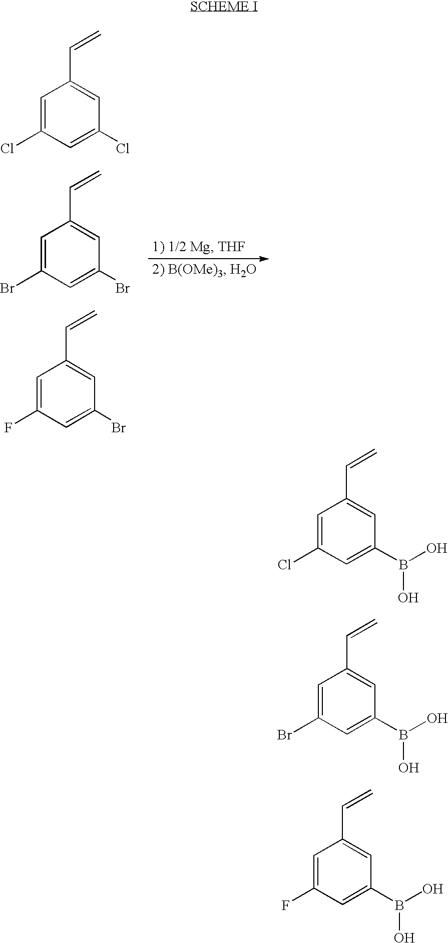

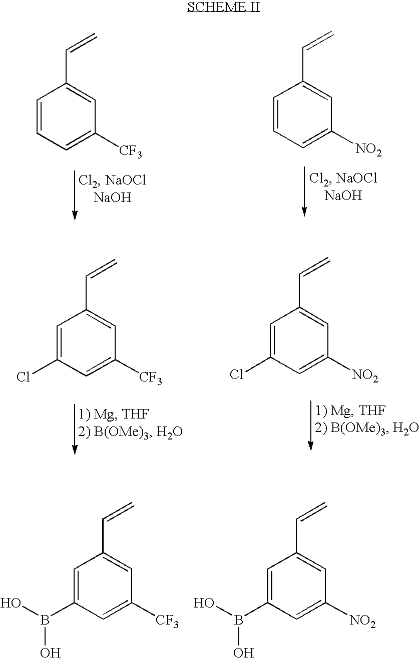

The boronic acid-containing polymers may include, in addition to the monomeric units derived from an ethylenically unsaturated monomer containing the boronic acid moiety, a monomeric unit derived from an ethylenically unsaturated monomer containing a reactive moiety. Specifically, the ethylenic unsaturation of this monomer renders the monomer copolymerizable with the boronic acid-containing monomer. In addition, this monomer contains a reactive moiety that is reactive with the biomedical device surface functional groups at the surface of the biomedical device as discussed hereinabove.

Representative examples of reactive monomers include, but are not limited to, ethylenically unsaturated carboxylic acids such as (meth)acrylic acid and the like; ethylenically unsaturated primary amines, such as 2-aminoethyl(meth)acrylate, N-(2-aminoethyl)(meth)acrylamide, 3-aminopropyl(meth)acrylate, N-(3-aminopropyl)(meth)acrylamide and the like; alcohol-containing (meth)acrylates and (meth)acrylamides such as 2-hydroxyethyl methacrylate and the like; ethylenically unsaturated epoxy-containing monomers such as glycidyl methacrylate, glycidyl vinyl carbonate and the like; and azlactone-containing monomers such as 2-isopropenyl-4,4-dimethyl-2-oxazolin-5-one, 2-vinyl-4,4-dimethyl-2-oxazolin-5-one and the like, where the azlactone group hydrolyzes in aqueous media to convert the oxazolinone moiety to a reactive carboxylic acid moiety.

The boronic acid-containing polymers can further include a monomeric unit containing a tertiary-amine moiety. Suitable monomers copolymerizable with the boronic acid monomer are ethylenically unsaturated monomers containing the tertiary-amine moiety. Representative examples include, but are not limited to, 2-(N,N-dimethyl)ethylamino(meth)acrylate, N-[2-(dimethylamino)ethyl](meth)acrylamide, N-[(3-dimethylamino)propyl](meth)acrylate, N-[3-dimethylamino)propyl](meth)acrylamide, vinylbenzyl-N,N-dimethylamine and the like and mixtures thereof.

The boronic acid-containing polymers may further include a hydrophilic monomeric unit. A suitable hydrophilic monomeric unit includes ethylenically unsaturated hydrophilic monomers that are copolymerizable with the boronic acid ethylenically unsaturated monomer. Representative examples include, but are not limited to, N,N-dimethylacrylamide, N,N-dimethylmethacrylamide and the like; cyclic lactams such as N-vinyl-2-pyrrolidone and the like; (meth)acrylated alcohols such as 2-hydroxyethyl methacrylate, 2-hydroxyethyl acrylate and the like; (meth)acrylated poly(ethyleneglycol)s and the like and mixtures thereof. The hydrophilic monomeric unit in the polymer, when used, ensures that the polymer is water-soluble, thus avoiding the need to dissolve the polymer in organic solvent when applying the polymer to the lens surface.

One class of boronic acid-containing polymers are copolymers containing at least monomeric units derived from an ethylenically unsaturated monomer containing one or more boronic acid moieties, and monomeric units derived from an ethylenically unsaturated monomer containing a moiety reactive with the complementary reactive functionalities at the surface of the biomedical device. These copolymers further include monomeric units derived from the ethylenically unsaturated monomer containing a tertiary-amine moiety, and monomeric units derived from an ethylenically unsaturated hydrophilic monomer in an amount sufficient to render the copolymer water soluble. This class of copolymers may contain about 1 to about 30 mole percent of the boronic acid-containing monomeric units, and preferably about 2 to about 20 mole percent; and about 2 to about 60 mole percent of monomeric units derived from an ethylenically unsaturated monomer containing the moiety reactive with complementary reactive functionalities at the surface of the biomedical device, and preferably about 5 to about 40 mole percent. In one embodiment, these copolymers contain at least 0 to about 50 mole percent of the tertiary-amine-containing monomeric units, and preferably about 5 to about 40 mole percent; and 0 to about 90 mole percent of the hydrophilic monomeric units, and preferably about 20 to about 80 mole percent.

Another class of polymers is copolymers containing at least monomeric units derived from an ethylenically unsaturated monomer containing one or more boronic acid moieties; monomeric units derived from the ethylenically unsaturated monomer containing the tertiary-amine moiety; and monomeric units derived from an ethylenically unsaturated hydrophilic monomer in an amount sufficient to render the copolymer water soluble. This class of copolymers may contain about 1 to about 30 mole percent of the boronic acid-containing monomeric units, and preferably about 2 to about 20 mole percent; and about 2 to about 50 mole percent of monomeric units derived from the ethylenically unsaturated tertiary-amine-containing monomeric units, and preferably about 5 to about 40 mole percent; and about 10 to about 90 mole percent of the hydrophilic monomeric units, and preferably about 20 to about 80 mole percent.

As discussed hereinabove, the polymers may include monomeric units derived from an ethylenically unsaturated monomer containing a reactive moiety which links the polymer to the surface of the biomedical device. One manner of linking the boronic acid-containing polymer to the surface of the biomedical device involves forming the device from a monomer mixture including a monomer that includes reactive functionalities that are complementary with the reactive moiety of the polymer.

As a first example, the biomedical device may be formed of the polymerization product of a monomer mixture comprising an epoxy-containing monomer, such as glycidyl methacrylate or glycidyl vinyl carbonate. Sufficient epoxy groups will migrate to the lens surface, and these epoxy groups covalently react with functionalities of the boronic acid-containing polymer, such as carboxylic acid, amino and alcohol reactive moieties.

As a second example, the biomedical device may be formed of the polymerization product of a monomer mixture comprising a carboxylic acid-containing monomer, such as (meth)acrylic acid or vinyl carbonic acid. Sufficient carboxylic groups will be present at the surface of the biomedical device to covalently react with functionalities of the boronic acid-containing polymer, such as amino and alcohol reactive moieties.

As a third example, the biomedical device may be formed of the polymerization product of a monomer mixture comprising an azlactone-containing monomer, such as 2-isopropenyl-4,4-dimethyl-2-oxazolin-5-one and 2-vinyl-4,4-dimethyl-2-oxazolin-5-one. Azlactone groups at the lens surface will hydrolyze in aqueous media to convert the oxazolinone group to a carboxylic acid, for reaction with the boronic acid-containing polymer reactive moieties.

As a fourth example, the biomedical device may be formed of the polymerization product of a monomer mixture comprising a (meth)acrylate or (meth)acrylamide alcohol, such as 2-hydroxyethyl methacrylate. The alcohol groups are available to react with boronic acid-containing polymer reactive moieties.

Other lens-forming monomers containing complementary reactive groups are known in the art, including those disclosed in U.S. Pat. No. 6,440,571, the contents of which are incorporated by reference herein.

Another manner of linking the boronic acid-containing polymer to the surface of the biomedical device involves treating the surface of the biomedical device to provide reactive functionalities on the surface that are complementary with the reactive moiety of the polymer. As an example, the surface of the biomedical device may be subjected to plasma treatment in an oxygen-containing atmosphere to form alcohol functionalities on the surface of the biomedical device, or in a nitrogen-containing atmosphere to form amine functionalities on the surface of the biomedical device. In the case that the biomedical device contains fluorine at its surface, the surface may be initially plasma treated in a hydrogen atmosphere to reduce fluorine content at the lens surface. Such methods are known in the art, including U.S. Pat. Nos. 6,550,915 and 6,794,456, the contents of which are incorporated by reference herein.

The alcohol or amino functionality generated at the surface by the plasma treatment may then react with reactive moieties of the boronic acid-containing polymer, such as carboxylic acid moieties.

A variation of plasma treatment involves initially subjecting the surface of the biomedical device to a plasma oxidation, followed by plasma polymerization in an atmosphere containing a hydrocarbon (such as a diolefin, for example, 1,3-butadiene) to form a carbon layer on the lens surface. Then, this carbon layer is plasma treated in an oxygen or nitrogen atmosphere to generate hydroxyl or amine radicals. The reactive moiety of the boronic acid-containing polymer can then be covalently attached to the hydroxyl or amine radicals of the carbon layer. See, e.g., U.S. Pat. No. 6,213,604, the contents of which are incorporated by reference herein.

In the case of silicone hydrogel contact lenses, the lenses may be plasma treated in an oxygen-containing atmosphere to form a silicate-containing surface on the lens, which surface then binds the boronic acid-containing polymer.

As used herein, the term “plasma treatment” is inclusive of wet or dry corona discharge treatments.

The hydrophilic hydrolyzed reactive polymers for attaching to the boronic acid-containing polymer are hydrophilic hydrolyzed reactive polymers comprising monomeric units derived from an ethylenically unsaturated-containing monomer having hydrolizable reactive functionalities. In general, the hydrophilic hydrolyzed reactive polymers are obtained by hydrolyzing a polymerization product of an ethylenically unsaturated-containing monomer having hydrolizable reactive functionalities, e.g., an epoxy group, by methods known in the art.

In one embodiment, an ethylenically unsaturated-containing monomer having hydrolizable reactive functionalities includes ethylenically unsaturated epoxy-containing monomers. Useful ethylenically unsaturated epoxy-containing monomers include glycidyl-containing ethylenically unsaturated monomers such as glycidyl methacrylate, glycidyl acrylate, glycidyl vinylcarbonate, glycidyl vinylcarbamate, vinylcyclohexyl-1,2-epoxide and the like.

In another embodiment, the hydrophilic hydrolyzed reactive polymers contains the ring-opening monomeric units derived from a ring-opening reactive monomers having an azlactone group represented by the following formula:

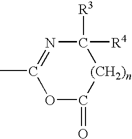

wherein R³ and R⁴ are independently an alkyl group having 1 to 14 carbon atoms, a cycloalkyl group having 3 to about 14 carbon atoms, an aryl group having 5 to about 12 ring atoms, an arenyl group having 6 to about 26 carbon atoms, and 0 to 3 heteroatoms non-peroxidic selected from S, N, and O, or R³ and R⁴ taken together with the carbon to which they are joined can form a carbocyclic ring containing 4 to 12 ring atoms, and n is an integer 0 or 1. Such monomeric units are disclosed in U.S. Pat. No. 5,177,165.

The ring structure of such reactive functionalities is susceptible to nucleophilic ring-opening reactions with complementary reactive functional groups on the surface of substrate being treated. For example, the azlactone functionality can react with primary amines, hydroxyl radicals or the like which may be present on the surface of the device to form a covalent bond between the substrate and the hydrophilic reactive polymer at one or more locations along the polymer. A plurality of attachments can form a series of polymer loops on the substrate, wherein each loop comprises a hydrophilic chain attached at both ends to the substrate.

Azlactone-functional monomers for making the hydrophilic hydrolyzed reactive polymer can be any monomer, prepolymer, or oligomer comprising an azlactone functionality of the above formula in combination with a vinylic group on an unsaturated hydrocarbon to which the azlactone is attached. Preferably, azlactone-functionality is provided in the hydrophilic polymer by 2-alkenyl azlactone monomers. The 2-alkenyl azlactone monomers are known compounds, their synthesis being described in, for example, U.S. Pat. Nos. 4,304,705; 5,081,197; and 5,091,489, the content of which are incorporated by reference herein. Suitable 2-alkenyl azlactones include, but are not limited to, 2-ethenyl-1,3-oxazolin-5-one, 2-ethenyl-4-methyl-1,3-oxazolin-5-one, 2-isopropenyl-1,3-oxazolin-5-one, 2-isopropenyl-4-methyl-1,3-oxazolin-5-one, 2-ethenyl-4,4-dimethyl-1,3-oxazolin-5-one, 2-isopropenyl-4,-dimethyl-1,3-oxazolin-5-one, 2-ethenyl-4-methyl-ethyl-1,3-oxazolin-5-one, 2-isopropenyl-4-methyl-4-butyl-1,3-oxazolin-5-one, 2-ethenyl-4,4-dibutyl-1,3-oxazolin-5-one, 2-isopropenyl-4-methyl-4-dodecyl-1,3-oxazolin-5-one, 2-isopropenyl-4,4-diphenyl-1,3-oxazolin-5-one, 2-isopropenyl-4,4-pentamethylene-1,3-oxazolin-5-one, 2-isopropenyl-4,4-tetramethylene-1,3-oxazolin-5-one, 2-ethenyl-4,4-diethyl-1,3-oxazolin-5-one, 2-ethenyl-4-methyl-4-nonyl-1,3-oxazolin-5-one, 2-isopropenyl-methyl-4-phenyl-1,3-oxazolin-5-one, 2-isopropenyl-4-methyl-4-benzyl-1,3-oxazolin-5-one, and 2-ethenyl-4,4-pentamethylene-1,3-oxazolin-5-one. In a preferred embodiment, the azlactone monomers are represented by the following general formula:

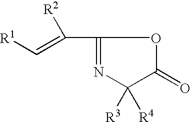

where R¹ and R² independently denote a hydrogen atom or a lower alkyl radical with one to six carbon atoms, and R³ and R⁴ independently denote alkyl radicals with one to six carbon atoms or a cycloalkyl radical with five or six carbon atoms. Specific examples include 2-isopropenyl-4,4-dimethyl-2-oxazolin-5-one (IPDMO), 2-vinyl-4,4-dimethyl-2-oxazolin-5-one (VDMO), spiro-4′-(2′-isopropenyl-2′-oxazolin-5-one) cyclohexane (IPCO), cyclohexane-spiro-4′-(2′-vinyl-2′-oxazol-5′-one) (VCO), and 2-(-1-propenyl)-4,4-dimethyl-oxazol-5-one (PDMO) and the like. These compounds and their preparation are known in the art, see, e.g., U.S. Pat. No. 6,858,310, the contents of which are incorporated by reference herein.

The hydrophilic hydrolyzed reactive polymers may further contain non-reactive hydrophilic monomeric units. Suitable hydrophilic non-reactive monomers include aprotic types or protic types or mixtures thereof. Suitable aprotic types include acrylamides such as N,N-dimethylacrylamide, N,N-dimethylmethacrylamide, N-methylmethacrylamide, N-methylacrylamide and the like, but preferably N,N-dimethylacrylamide for increased hydrophilicity; lactams such as N-vinylpyrrolidinone and the like, poly(alkylene oxides) such as methoxypolyoxyethylene methacrylates and the like and mixtures thereof. Suitable protic types include methacrylic acid, hydroxyalkyl(meth)acrylates such as 2-hydroxyethyl methacrylate and the like and mixtures thereof.

If desired, the copolymers may further include monomeric units which are hydrophobic optionally may be used in amounts up to 35 mole percent, preferably 0 to 20 mole percent, most preferably 0 to 10 mole percent. Examples of hydrophobic monomers are alkyl methacrylate, fluorinated alkyl methacrylates, long-chain acrylamides such as octyl acrylamide, and the like.

Generally, the hydrophilic hydrolyzed reactive polymers comprise about 1 to about 100 mole percent of reactive ethylenically unsaturated hydrolyzed epoxy-containing monomeric units, and preferably about 5 to about 50 mole percent, and more preferably about 10 to about 40 mole percent. The polymers may further contain 0 to about 99 mole percent of non-reactive hydrophilic monomeric units, preferably about 50 to about 95 mole percent, more preferably about 60 to about 90 mole percent (the reactive monomers, once reacted may also be hydrophilic, but are by definition mutually exclusive with the monomers referred to as hydrophilic monomers which are non-reactive).

The boronic acid-containing monomers and hydrophilic hydrolyzed reactive polymers can be synthesized in a manner known per se from the corresponding monomers (the term monomer here also including a macromer) by a polymerization reaction customary to the person skilled in the art. Typically, the polymers or chains are formed by subjecting a monomer(s)/photoinitiator mixture to a source of ultraviolet or actinic radiation and/or elevated temperature and curing the mixture. Typical polymerization initiators include free-radical-generating polymerization initiators such as acetyl peroxide, lauroyl peroxide, decanoyl peroxide, caprylyl peroxide, benzoyl peroxide, tertiary butyl peroxypivalate, sodium percarbonate, tertiary butyl peroctoate, and azobis-isobutyronitrile (AIBN). Typical ultraviolet free-radical initiators such as diethoxyacetophenone can also be used. The curing process will of course depend upon the initiator used and the physical characteristics of the monomer or monomer mixture such as viscosity. In any event, the level of initiator employed will vary within the range of about 0.001 to about 2 weight percent of the mixture of monomers.

Polymerization to form the resulting boronic acid-containing polymers and hydrophilic hydrolyzed reactive polymers can be carried out in the presence or absence of a solvent. Suitable solvents are in principle all solvents which dissolve the monomer used, e.g., water; alcohols such as lower alkanols, for example, ethanol and methanol; carboxamides such as dimethylformamide, dipolar aprotic solvents such as dimethyl sulfoxide or methyl ethyl ketone; ketones such as acetone or cyclohexanone; hydrocarbons such as toluene; ethers such as tetrahydrofuran, dimethoxyethane or dioxane; halogenated hydrocarbons such as trichloroethane, and also mixtures of suitable solvents, for example mixtures of water and an alcohol such as water/methanol or a water/ethanol mixture.

In general, a method of making the surface modified biomedical device of the present invention involves exposing a biomedical device having a plurality of biomedical device surface functional groups to (a) one or more polymers comprising monomeric units derived from an ethylenically unsaturated monomer containing one or more boronic acid moieties and; and (b) a hydrophilic hydrolyzed reactive polymer comprising monomeric units derived from an ethylenically unsaturated-containing monomer having hydrolyzable reactive functionalities, thus forming a biocompatible surface on the biomedical device. In one embodiment, a method of making the surface modified biomedical device of the present invention involves covalently bonding the one or more polymers comprising monomeric units derived from at least an ethylenically unsaturated monomer containing one or more boronic acid moieties to the surface of the biomedical device to form an inner layer via reaction with the biomedical device surface functional groups of the biomedical device by techniques known in the art.

For example, the biomedical device can be contacted with a solution containing the one or more polymers comprising monomeric units derived from at least an ethylenically unsaturated monomer containing one or more boronic acid moieties to the biomedical device surface functional groups of the biomedical device for a time period sufficient to form an inner layer on the surface of the biomedical device.

Next, the hydrophilic hydrolyzed reactive polymer comprising monomeric units derived from an ethylenically unsaturated-containing monomer having hydrolizable reactive functionalities is exposed to the biomedical device having an inner layer, e.g., as a solution, on the surface thereof thereby forming an outer layer on the surface on the biomedical device.

In another embodiment, a method of making the surface modified biomedical devices of the present invention involves (a) placing in a biomedical device package the biomedical device and a solution comprising the polymer comprising monomeric units derived from an ethylenically unsaturated monomer containing one or more boronic acid moieties and a hydrophilic hydrolyzed reactive polymer comprising monomeric units derived from an ethylenically unsaturated-containing monomer having hydrolizable reactive functionalities; (b) sealing the package with lidstock; and (c) autoclaving the package and its contents.

Preferably, the outer layer is removed from the inner layer while the biomedical device is worn and replaced with epithelial mucin. Preferably, the boronic acid-containing polymer has greater affinity to mucin than does the hydrophilic hydrolyzed reactive polymer, and the boronic acid-containing polymer has greater affinity to the surface of the biomedical device than does the hydrophilic hydrolyzed reactive polymer. In one embodiment, the boronic acid-containing polymer is permanently bound to the contact lens, and the hydrophilic hydrolyzed reactive polymer is temporarily bound to the boronic acid-containing polymer.

The following examples are provided to enable one skilled in the art to practice the invention and are merely illustrative of the invention. The examples should not be read as limiting the scope of the invention as defined in the claims.

In the examples, the following abbreviations are used.

APMA: 3-aminopropylmethacrylamide. HCl

AEMA: 2-aminoethyl methacrylate

DMAEMA: N-[(2-dimethylamino)ethyl]methacrylate

DMAPMA: N-[(3-dimethylamino)propyl]methacrylamide

MAAPBA: 3-methacrylamidophenylboronic acid

SBA: 4-vinylphenylboronic acid

MAA: methacrylic acid

GM: glycidyl methacrylate

DMA: N,N-dimethylacrylamide

NVP: N-vinyl-2-pyrrolidone

OFPMA: 1H,1H,5H-octafluoropentylmethacrylate

LMA: laurylmethacrylate

VCHE: 4-vinylcyclohexyl-1,2-epoxide

THF: tetrahydrofuran

AIBN: a thermal polymerization initiator, said to be 2,2′-azobisisobutyronitrile (DuPont Chemicals, Wilmington, Del.) and known as Vazo™ 64

### Example 1

### Synthesis of a Copolymer of N,N-dimethylacrylamide-co-1H,1H,5H-octafluoropentylmethacrylate-co-glycidyl Methacrylate

To a 3000 ml reaction flask were added distilled DMA (128 g, 1.28 moles), OFPMA (8 g, 0.024 moles, used as received), distilled GM (32 g, 0.224 moles), AIBN (0.24 g, 0.00144 moles) and tetrahydrofuran (2000 ml). The reaction vessel was fitted with a magnetic stirrer, condenser, thermal controller and a nitrogen inlet. Nitrogen was bubbled through the solution for 15 minutes to remove any dissolved oxygen. The reaction flask was then heated to 60° C. under a passive blanket of nitrogen for 20 hours. The reaction mixture was then added slowly to 12 L of ethyl ether with good mechanical stirring. The reactive polymer precipitated and was collected by vacuum filtration. The solid was placed in a vacuum oven at 30° C. overnight to remove the ether leaving 113 g of reactive polymer (67% yield). The reactive polymer was placed in a desiccator for storage until use.

### Example 2

### Hydrolysis of Epoxide Groups on the Copolymer of Example 1

The copolymer of Example 1 (2.59 g) was dissolved in purified water (80 ml), in a sealed jar and placed in an oven at 60° C. for five days. The water was removed by freeze drying and a sample of the recovered copolymer was analyzed by C¹³ nuclear magnetic resonance (NMR) spectroscopy. The sample showed no evidence of glycidyl groups confirming complete hydrolysis of the epoxy groups to 1,3 diols. A total of 2.4 grams of copolymer was isolated after the hydrolysis reaction.

### Example 3

### Synthesis of a Boronic Acid-Containing Polymer

To a 1-L 3-neck round bottom flask containing a magnetic stir bar, water-cooled condenser and thermocouple is added approximately 0.2-wt % AIBN initiator (based on total weight of monomers), 5.0-mol % of SBA, 10-mol % of MAA, 20-mol % of DMAPMA and 65-mol % of DMA. The monomers and initiator are dissolved by addition of 300-mL of methanol to the flask. The solution is sparged with argon for at least 10-min. before gradual heating to 60° C. Sparging is discontinued when the solution reaches 40 to 45° C. and the flask is subsequently maintained under argon backpressure. Heating is discontinued after 48 to 72 hours at which point the cooled solution is added dropwise to 6 L of mechanically stirred ethyl ether. The precipitate is isolated either by filtration or decanting off the ether. The solid is dried in vacuo at 80° C. for a minimum of 18 hours and reprecipitated by dissolution in 300-mL methanol and dropwise addition into 6-L of stirred ethyl ether. The final polymer mass is determined after vacuum drying at 80° C. to a constant mass.

### EXAMPLES 4-17

### Synthesis of a Boronic Acid-Containing Polymer

The polymers of Examples 4-17 were synthesized in substantially the same manner as Example 3. The ingredients and amounts used are set forth below in Table 1.

TABLE 1

| | Ex 4 | Ex 5 | Ex 6 | Ex 7 | Ex 8 | Ex 9 | Ex 10 |
|---|---|---|---|---|---|---|---|
| DMA (mol %) | 65 | 50 | 55 | 40 | 65 | 68.5 | 70 |
| DMAPMA (mol %) | 20 | 30 | 25 | 20 | — | 19 | — |
| DMAEMA (mol %) | — | — | — | — | 20 | — | 20 |
| MAA (mol %) | 10 | 10 | 10 | 30 | 10 | — | — |
| APMA (mol %) | — | — | — | — | — | 7.5 | — |
| SBA (mol %) | 5 | 10 | 10 | 10 | 5 | 5 | 5 |
| AEMA (mol %) | — | — | — | — | — | — | 5 |
| | Ex 11 | Ex 12 | Ex 13 | Ex 14 | Ex 15 | Ex 16 | Ex 17 |
| DMA (mol %) | 70 | 70 | 65 | 65 | 70 | 85 | 85 |
| DMAPMA (mol %) | 20 | 20 | 15 | 10 | 16 | 10 | 10 |
| MAA (mol %) | — | 7.5 | — | — | 7 | — | — |
| APMA (mol %) | 7.5 | — | 10 | 10 | — | — | — |
| SBA (mol %) | — | — | 10 | 15 | 7 | 5 | — |
| MAAPBA (mol %) | 2.5 | 2.5 | — | — | — | — | 5 |

Examples 18-24 illustrate the syntheses of hydrophilic hydrolyzed reactive polymers that may be used to link to the boronic acid moieties of the lens surface.

### Example 18

Copolymer of DMA/GMA (86/14 mol/mol).

To a 1 L reaction flask were added distilled DMA (48 g, 0.48 moles), distilled GMA (12 g, 0.08 moles), AIBN (0.1 g, 0.0006 moles) and anhydrous THF (500 ml). The reaction vessel was fitted with a mechanical stirrer, condenser, thermal controller and a nitrogen inlet. Nitrogen was bubbled through the solution for 15 minutes to remove any dissolved oxygen. The reaction flask was then heated to 40° C. under a passive blanket of nitrogen for 168 hours. The reaction mixture was then added slowly to ethyl ether (1.5 L) with good mechanical stirring. The reactive polymer precipitated and organic solvents were decanted. The solid was collected by filtration and placed in a vacuum oven to remove the ether leaving 58.2 g of reactive polymer (97% yield). The resulting copolymer is hydrolyzed in substantially the same manner as the copolymer in Example 2.

### Example 19

Copolymer of DMA/GMA (76/24 mol/mol).

To a 1 L reaction flask were added distilled DMA (42 g, 0.42 moles), distilled GMA (18 g, 0.13 moles), AIBN (0.096 g, 0.0006 moles) and toluene (600 ml). The reaction vessel was fitted with a magnetic stirrer, condenser, thermal controller and a nitrogen inlet. Nitrogen was bubbled through the solution for 15 minutes to remove any dissolved oxygen. The reaction flask was then heated to 60° C. under a passive blanket of nitrogen for 20 hours. The reaction mixture was then added slowly to 6 L of ethyl ether with good mechanical stirring. The reactive polymer precipitated and was collected by vacuum filtration. The solid was placed in a vacuum oven at 30° C. overnight to remove the ether leaving 46.7 g of reactive polymer (78% yield). The resulting copolymer is hydrolyzed in substantially the same manner as the copolymer in Example 2.

### Example 20

Copolymer of DMA/GMA (68/32 mol/mol).

To a 1 L reaction flask were added distilled DMA (36 g, 0.36 moles), distilled GMA (24 g, 0.17 moles), AIBN (0.096 g, 0.0006 moles) and toluene (600 ml). The reaction vessel was fitted with a magnetic stirrer, condenser, thermal controller and a nitrogen inlet. Nitrogen was bubbled through the solution for 15 minutes to remove any dissolved oxygen. The reaction flask was then heated to 60° C. under a passive blanket of nitrogen for 20 hours. The reaction mixture was then added slowly to 6 L of ethyl ether with good mechanical stirring. The reactive polymer precipitated and was collected by vacuum filtration. The solid was placed in a vacuum oven at 30° C. overnight to remove the ether leaving 49.8 g of reactive polymer (83% yield). The resulting copolymer is hydrolyzed in substantially the same manner as the copolymer in Example 2.

### Example 21

Copolymer of DMA/OFPMA/GMA (84/1.5/14.5 mol/mol/mol)

To a 3000 ml reaction flask were added distilled DMA (128 g, 1.28 moles), OFPMA (8 g, 0.024 moles), distilled GMA (32 g, 0.224 moles), AIBN (0.24 g, 0.00144 moles) and THF (2000 ml). The reaction vessel was fitted with a magnetic stirrer, condenser, thermal controller and a nitrogen inlet. Nitrogen was bubbled through the solution for 15 minutes to remove any dissolved oxygen. The reaction flask was then heated to 60° C. under a passive blanket of nitrogen for 20 hours. The reaction mixture was then added slowly to 12 L of ethyl ether with good mechanical stirring. The reactive polymer precipitated and was collected by vacuum filtration. The solid was placed in a vacuum oven at 30° C. overnight to remove the ether leaving 134.36 g of reactive polymer (80% yield). The resulting copolymer is hydrolyzed in substantially the same manner as the copolymer in Example 2.

### Example 22

Copolymer of DMA/OFPMA/GMA (85/0.18/14.82 mol/mol/mol).

To a 500 ml reaction flask were added distilled DMA (16 g, 0.16 moles), OFPMA (0.1 g, 0.0003 moles, used as received), distilled GMA (4 g, 0.028 moles), AIBN (0.063 g, 0.00036 moles) and THF (300 ml). The reaction vessel was fitted with a magnetic stirrer, condenser, thermal controller and a nitrogen inlet. Nitrogen was bubbled through the solution for 15 minutes to remove any dissolved oxygen. The reaction flask was then heated to 60° C. under a passive blanket of nitrogen for 20 hours. The reaction mixture was then added slowly to 3 L of ethyl ether with good mechanical stirring. The reactive polymer precipitated and was collected by vacuum filtration. The solid was placed in a vacuum oven at 30° C. overnight to remove the ether leaving 14.5 g of reactive polymer (69 yield). The resulting copolymer is hydrolyzed in substantially the same manner as the copolymer in Example 2.

### Example 23

Copolymer of DMA/LMA/GMA (84/1.5/14.5 mol/mol/mol)

To a 1000 ml reaction flask were added distilled DMA (32 g, 0.32 moles), LMA (1.5 g, 0.006 moles, used as received), distilled GMA (8 g, 0.056 moles), AIBN (0.06 g, 0.00036 moles) and THF (600 ml). The reaction vessel was fitted with a magnetic stirrer, condenser, thermal controller and a nitrogen inlet. Nitrogen was bubbled through the solution for 15 minutes to remove any dissolved oxygen. The reaction flask was then heated to 60° C. under a passive blanket of nitrogen for 20 hours. The reaction mixture was then added slowly to 3 L of ethyl ether with good mechanical stirring. The reactive polymer precipitated and was collected by vacuum filtration. The solid was placed in a vacuum oven at 30° C. overnight to remove the ether leaving 29.2 g of reactive polymer (70% yield). The resulting copolymer is hydrolyzed in substantially the same manner as the copolymer in Example 2.

### Example 24

Copolymer of NVP/VCHE (85/15 mol/mol).

To a 1 L reaction flask were added distilled NVP (53.79 g, 0.48 moles), VCHE (10.43 g, 0.084 moles), AIBN (0.05 g, 0.0003 moles) and THF (600 ml). The reaction vessel was fitted with a magnetic stirrer, condenser, thermal controller and a nitrogen inlet. Nitrogen was bubbled through the solution for 15 minutes to remove any dissolved oxygen. The reaction flask was then heated to 60° C. under a passive blanket of nitrogen for 20 hours. The reaction mixture was then added slowly to 6 L of ethyl ether with good mechanical stirring. The copolymer precipitated and was collected by vacuum filtration. The solid was placed in a vacuum oven at 30° C. overnight to remove the ether leaving 21 g of reactive polymer (a 32% yield). The reactive polymer was placed in a desiccator for storage until use. The resulting copolymer is hydrolyzed in substantially the same manner as the copolymer in Example 2.

### Example 25

### Coating of Contact Lenses with Boronic Acid-Containing Polymers

Contact lenses made of Balafilcon A are cast and processed under standard manufacturing procedures. Balafilcon A is a copolymer comprised of 3-[tris(tri-methylsiloxy)silyl]propyl vinyl carbamate, N-vinyl-2-pyrrolidone (NVP), 1,3-bis[4-vinyloxycarbonyloxy)but-1-yl]polydimethylsiloxane and N-vinyloxycarbonyl alanine. The Balafilcon A lenses are air-plasma treated.

For coating with the boronic acid-containing polymers of Examples 3-17, each lens in placed in a vial containing a boronic acid-containing polymer of Examples 3-17 dissolved in deionized water or phosphate buffered saline. The vials are capped and placed in a forced-air oven heated to 90° C. for 2 hours. Next, the lenses are removed from the vials and placed in polypropylene contact lens blister packs containing a buffered saline solution of a hydrophilic hydrolyzed reactive polymer of Examples 2 and 18-24. The blisters are sealed and autoclaved at 121° C. for 30 minutes.

It will be understood that various modifications may be made to the embodiments disclosed herein. Therefore the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention. Moreover, those skilled in the art will envision other modifications within the scope and spirit of the features and advantages appended hereto.

What is claimed is:

1. A surface modified biomedical device having a coating on a surface thereof, the coating comprising an inner layer comprising a polymer comprising monomeric units derived from an ethylenically unsaturated monomer containing one or more boronic acid moieties, and an outer layer comprising a hydrophilic hydrolyzed reactive polymer comprising monomeric units derived from an ethylenically unsaturated containing monomer having hydrolyzable reactive functionalities.

2. The surface modified biomedical device of claim 1, wherein the ethylenically unsaturated monomer containing one or more boronic acid moieties is an ethylenically unsaturated containing aryl boronic acid.

3. The surface modified biomedical device of claim 1, wherein the ethylenically unsaturated monomer containing one or more boronic acid moieties is selected from the group consisting of 4-vinylphenylboronic acid, 3-methacrylamidophenylboronic acid and mixtures thereof.

4. The surface modified biomedical device of claim 1, wherein the polymer comprising monomeric units derived from an ethylenically unsaturated monomer containing one or more boronic acid moieties is a copolymer comprising monomeric units derived from an ethylenically unsaturated monomer containing one or more boronic acid moieties; and monomeric units derived from an ethylenically unsaturated monomer containing a moiety reactive with biomedical device surface functional groups at the surface of the biomedical device.

5. The surface modified biomedical device of claim 4, wherein the biomedical device surface functional group is selected from the group consisting of a hydroxy group, amino group, carboxy group, carbonyl group, aldehyde group, sulfonic acid group, sulfonyl chloride group, isocyanato group, carboxy anhydride group, lactone group, azlactone group, epoxy group and mixtures thereof.

6. The surface modified biomedical device of claim 1, wherein the polymer comprising monomeric units derived from an ethylenically unsaturated monomer containing one or more boronic acid moieties is a copolymer comprising monomeric units derived from an ethylenically unsaturated monomer containing one or more boronic acid moieties; and monomeric units derived from an ethylenically unsaturated monomer containing a tertiary-amine moiety.

7. The surface modified biomedical device of claim 1, wherein the hydrophilic hydrolyzed reactive polymer is a copolymer comprising monomeric units derived from an ethylenically unsaturated monomer containing epoxy groups.

8. The surface modified biomedical device of claim 1, wherein the hydrophilic hydrolyzed reactive polymer is a copolymer obtained from a hydrolyzed polymerization product of a monomer mixture comprising an ethylenically unsaturated epoxy-containing monomer.

9. The surface modified biomedical device of claim 8, wherein the ethylenically unsaturated epoxy-containing monomer is selected from the group consisting of glycidyl methacrylate, glycidyl acrylate, glycidyl vinylcarbonate, glycidyl vinylcarbamate, vinylcyclohexyl-1,2-epoxide and mixtures thereof.

10. The surface modified biomedical device of claim 1, wherein the hydrophilic hydrolyzed reactive polymer comprises ring-opening monomeric units derived from a ring-opening reactive monomer having an azlactone group.

11. The surface modified biomedical device of claim 10, wherein the hydrophilic hydrolyzed reactive polymer further comprises monomeric units derived from an aprotic hydrophilic monomer selected from the group consisting of N,N-dimethylacrylamide, N,N-dimethyl methacrylamide, N-methylmethacrylamide, N-methylacrylamide; N-vinylpyrrolidinone, methoxypolyoxyethylene methacrylates and mixtures thereof.

12. The surface modified biomedical device of claim 10, wherein the hydrophilic hydrolyzed reactive polymer further comprises monomeric units derived from a protic hydrophilic monomer is selected from the group consisting of methacrylic acid, 2-hydroxyethyl methacrylate and mixtures thereof.

13. The surface modified biomedical device of claim 1, wherein the inner layer is covalently linked to the surface of the biomedical device through primary amine or hydroxyl radicals at the surface of the device.

14. The surface modified biomedical device of claim 1, wherein the biomedical device is an ophthalmic lens.

15. The surface modified biomedical device of claim 14, wherein the ophthalmic lens is a contact lens or an intraocular lens.

16. A method for making a surface modified biomedical device, the method comprising exposing a biomedical device having a plurality of biomedical device surface functional groups to (a) one or more polymers comprising monomeric units derived from an ethylenically unsaturated monomer containing one or more boronic acid moieties; and (b) a hydrophilic hydrolyzed reactive polymer comprising monomeric units of an ethylenically unsaturated-containing monomer having hydrolyzable reactive functionalities, thus forming a biocompatible coating on the surface on the biomedical device.

17. The method of claim 16, wherein the biocompatible coating on the surface comprises an inner layer comprising the polymer comprising monomeric units derived from an ethylenically unsaturated monomer containing one or more boronic acid moieties, and an outer layer comprising the hydrophilic hydrolyzed reactive polymer comprising monomeric units derived from an ethylenically unsaturated containing monomer having hydrolyzable reactive functionalities.

18. The method of claim 16, wherein the ethylenically unsaturated monomer containing one or more boronic acid moieties is selected from the group consisting of 4-vinylphenylboronic acid, 3-methacrylamidophenylboronic acid and mixtures thereof and the hydrophilic hydrolyzed reactive polymer is a copolymer comprising monomeric units derived from an ethylenically unsaturated monomer containing epoxy groups.

19. The method of claim 16, wherein the ethylenically unsaturated monomer containing one or more boronic acid moieties is selected from the group consisting of 4-vinylphenylboronic acid, 3-methacrylamidophenylboronic acid and mixtures thereof and the hydrophilic hydrolyzed reactive polymer comprises ring-opening monomeric units derived from a ring-opening reactive monomer having an azlactone group.

20. The method of claim 16, comprising
placing in a biomedical device package the biomedical device and a solution comprising the polymer comprising monomeric units derived from an ethylenically unsaturated monomer containing one or more boronic acid moieties and a hydrophilic hydrolyzed reactive polymer comprising monomeric units of an ethylenically unsaturated-containing monomer having hydrolyzable reactive functionalities;
sealing the package with lidstock; and
autoclaving the package and its contents.
